(19) United States
(12) Patent Application Publication
(10) Pub. No.: US 2008/0241865 A1
(43) Pub. Date: Oct. 2, 2008

(54) VISIBLE TO NEAR-INFRARED LIGHT PROBE COMPRISING COMPLEX OF CYPRIDINA LUCIFERASE AND QUANTUM DOT

(75) Inventors: Yoshihiro OHMIYA, Ikeda-shi (JP); Chun WU, Ikeda-shi (JP)

Correspondence Address: Warren M. Cheek, Jr.;WENDEROTH, LIND & PONACK, L.L.P., Suite 800, 2033 K Street, N.W., Washington, DC, 20006, US

(73) Assignee: NATIONAL INSTITUTE OF ADVANCED INDUSTRIAL SCIENCE AND TECHNOLOGY, Tokyo-to (JP)

(21) Appl. No.: 12/055,573

(22) Filed: Mar. 26, 2008

Related U.S. Application Data
(60) Provisional application No. 60/907,234, filed on Mar. 26, 2007.

(51) Int. Cl. C12Q 1/66

(52) U.S. Cl. 435/8

(57) ABSTRACT

The invention relates to a bioactive substance labeled with Cypridina luciferase and a quantum dot, the bioactive substance being at least one species selected from the group consisting of antibodies, peptides, organic compounds, hormones, enzyme substrates, sugar chains and nucleic acids.

This application claims the benefit of U.S. provisional application No. 60/907,234, filed Mar. 26, 2007, now abandoned.

### TECHNICAL FIELD

The present invention relates to a visible to near-infrared light probe comprising a Cypridina luciferase-quantum dot complex and Cypridina luciferin, and an analog thereto.

### BACKGROUND ART

In the field of life sciences, it is very important to analyze various cellular phenomena such as changes in the amount of cellular calcium, phosphorylation of intracellular proteins, distribution of ATP, which is a source of energy, transcriptional activities of genes and the like. Various molecular probes are made for such analytical means, and used for imaging. Such imaging techniques are also used for observations from the living cell level to the individual level. For example, labeling and imaging cancer cells enables the evaluation of anticancer agents and visual analysis of cancer metastasis. Radioactive isotope (RI), fluorescence, and luminescence probes are used for individual imaging probes. However, radioactive probes can be used only by qualified researchers in strictly controlled facilities, the viable life of the probe is determined by the half life of the isotope used, and measuring devices and probes are expensive. On the other hand, luminescence and fluorescence probes are not required to be used in controlled facilities, and do not require expensive measuring devices. Luminescence and fluorescence probes are stable and inexpensive, and easily handled. However, since fluorescence probes require excitation light, it is difficult to obtain information from deep inside individuals where external light sources cannot be used for excitation. Cellular photodamage caused by external light can also be problematic. Luminescence probes are self luminescent, and do not require excitation light; however, probes that can produce red to near-infrared light with high penetration efficiency at depth in individuals have not yet been developed.

Luciferase probes are one type of luminescence probes. Among luciferases, beetle luciferase is already utilized for intracellular imaging, and is found to be useful as a visualizing probe for use over a long period of time (WO2007/058140, WO2006/106752). Individual imaging devices for mice are available, and utilized for visualizing cancer tissues and the like.

### DISCLOSURE OF THE INVENTION

### Problems to be Solved by the Invention

An object of the present invention is to provide a luminescence probe that efficiently emits visible to near-infrared light that is excellent for individual imaging and use thereof.

Another object of the present invention is to provide a system in which luciferase can emit high energy visible light to ultraviolet light in a luciferase-quantum dot complex, and leading efficient excitation of the quantum dot.

### Means for Solving the Problems

The present inventors conducted extensive research to solve the above problems, and, as a result, found that excitation light with high energy can be produced by constructing a complex containing Cypridina luciferase and a quantum dot, and using luciferin or analogue thereof, thereby accomplishing the present invention.

The present invention provides a labeled bioactive substance, a method of producing thereof, a biological imaging method, and a cell labeling complex as shown below.

1. A bioactive substance labeled with Cypridina luciferase and a quantum dot, the bioactive substance being at least one species selected from the group consisting of antibodies, peptides, organic compounds, hormones, enzyme substrates, sugar chains and nucleic acids.

2. The labeled bioactive substance according to Item 1, wherein the bioactive substance, the Cypridina luciferase and the quantum dot form a complex through binding of an avidin substrate and biotin.

3. A biological imaging method, comprising applying a bioactive substance labeled with Cypridina luciferase and a quantum dot to a living body, the bioactive substance being at least one species selected from the group consisting of antibodies, peptides, organic compounds, hormones, enzyme substrates, sugar chains and nucleic acids.

4. A method of producing a bioactive substance complex labeled with Cypridina luciferase and a quantum dot, comprising the steps of preparing a complex of a polyvalent avidin substrate and a bioactive substance, and adding a biotinylated quantum dot and biotinylated Cypridina luciferase.

5. A method of producing a bioactive substance complex labeled with Cypridina luciferase and a quantum dot, comprising the steps of generating a complex of a polyvalent avidin substrate and Cypridina luciferase, and adding a biotinylated quantum dot and a biotinylated bioactive substance.

6. A cell labeling complex, comprising a Cypridina luciferase-bioactive substance fusion protein and a quantum dot, the bioactive substance being at least one species selected from the group of an antibody, a peptide, an organic compound, a hormone, an enzyme substrate, a sugar chain and a nucleic acid.

### Effects of the Invention

According to the present invention, Cypridina luciferase can emit visible to ultraviolet light containing light, thereby efficiently exciting the quantum dot. Cypridina luciferase can be fused with peptides or proteins (for example, antibodies, antigens, haptens, hormones, and the like), and accumulated in cancer tissues and the like. Therefore, cancer tissues can be individually imaged using near-infrared light, thereby enabling application to the treatment of various types of pathoses and the development of new drugs.

In transgenic cells introduced with the previously known firefly luminescence enzyme, the amount of luminescence emitted was not sufficient for luminescence imaging using near-infrared light. In contrast, a Cypridina luciferase-quantum dot complex of the present invention, when introduced into a living body, can efficiently emit visible to near-infrared light, thereby enabling the imaging of organs deeper inside individuals compared with previously used Renilla luciferase-quantum dot complexes.

### BRIEF DESCRIPTION OF DRAWINGS

FIG. 1 is a schematic drawing showing complex of Biotinylated Antibody, Biotinylated Luciferase (Biotin Enzyme) and Avidin Quantum Dot.

FIG. 2 is a graph showing luminescence by energy transfer in the longer wavelength region as observed in Example 2.

FIG. 3 is a graph showing luminescence observed in Example 3.

FIG. 4 is luminescence by energy transfer in the near-infrared region as observed in Example 4.

FIG. 5 shows emission spectrum measured in Example 5.

FIG. 6 shows emission spectrum measured in Example 6.

### BEST MODE FOR CARRYING OUT THE INVENTION

In one of the preferred embodiments of the present invention, the amount of luminescence emitted from the Cypridina luciferase-quantum dot complex and luciferin is, for example, at least 2 times, preferably at least 4 times, more preferably at least 10 times, more compared with the amount of luminescence emitted from the combination of firefly luciferase and a quantum dot.

The Cypridina luciferase used in the present invention is publicly known. In the specification and claims of the present invention, the term “Cypridina luciferase” generally includes wild type Cypridina luciferase and any mutants thereof. The amino acid sequence of the wild-type Cypridina luciferase is recorded in AAB86460, AAA30332, BAD08210, and the like.

Cypridina luciferase mutants may have a substitution, an addition, a deletion or an insertion of one or a few amino acids, preferably one or multiple amino acids, including optional mutants that possess the activity to emit luminescence from Cypridina luciferin as a substrate. The mutants have a homology of at least 70% to the above wild-type Cypridina luciferase, preferably at least 80%, more preferably at least 90%, even more preferably 95%, and particularly even more preferably at least 98% homology.

In the present invention, biotin may be bound to Cypridina luciferase through an amino group (amino group of N terminus or Lysine), a guanidino group (Arginine), a thiol group (Cysteine) of Cypridina luciferase, and is preferably bound through an amino group. The biotin can also possibly be bound through a sugar chain.

The preferable amino acid residues that biotin bind to are K180 and/or K203 of the amino acid sequence of AAB86460, and in the case of other Cypridina luciferases, the amino acid group of Lysine at the corresponding position is biotinylated.

The amino acid sequence of Cypridina luciferase (SEQ ID NO: 1) is presented below (K180 and/or K203 is underlined):

MRFPSIFTAVLFAASSALAALVNTTTEDETAQIPAEAVIGYSDLEGDFDV
AVLPFSNSTNNGLLFINTTIASIAAKEEGVSLEKREAEAQDCPYEPDPPN
TVPTSCEAKEGECIDSSCGTCTRDILSDGLCENKPGKTCCRMCQYVIECR
VEAAGWFRTFYGKRFQFQEPGTYVLGQGTKGGDWKVSITLENLDGTKGAV
LTKTRLEVAGDIIDIAQATENPITVNGGADPIIANPYTIGEVTIAVVEMP
GFNITVIEFFKLIVIDILGGRSVRIAPDTANKGMISGLCGDLKMMEDTDF
TSDPEQLAIQPKINQEFDGCPLYGNPDDVAYCKGLLEPYKDSCRNPINFY
YYTISCAFARCMGGDERASHVLLDYRETCAAPETRGTCVLSGHTFYDTFD
KARYQFQGPCKEILMAADCFWNTWDVKVSHRNVDSYTEVEKVRIRKQSTV
VELIVDGKQILVGGEAVSVPYSSQNTSIYWQDGDILTTAILPEALVVKFN
FKQLLVVHIRDPFDGKTCGICGNYNQDFSDDSFDAEGACDLTPNPPGCTE
EQKPEAERLCNSLFAGQSDLDQKCNVCHKPDRVERCMYEYCLRGQQGFCD
HAWEFKKECYIKHGDTLEVPDECKGSGSGSHHHHHH

Polyvalent avidin substrates used in the present invention may be any polyvalent avidin substrate that can link biotinylated Cypridina luciferase to a biotinylated bioactive substance and a biotinylated quantum dot, and preferable examples include streptavidin, NeutrAvidin® and avidin. In addition, the polyvalent avidin substrates generally include substrates that can bind to multiple biotin molecules, such as fusion proteins comprising multiple avidin molecules bound together.

### I. Biotin Binding to Cypridina Luciferase (Polypeptide)

Cypridina luciferase and biotin residues are linked through a spacer having a polyalkylene glycol structure. Examples of polyalkylene glycols include polyethylene glycol (PEG), polypropylene glycol (PPG), polybutylene glycol (PBG), (PEG)-(PPG)-(PEG) block copolymer, (PPG)-(PEG)-(PPG) block copolymer, (PEG)-(PBG)-(PEG) block copolymer, (PBG)-(PEG)-(PBG) block copolymer, and the like. Among them, PEG, PPG, (PEG)-(PPG)-(PEG) block copolymer and (PPG)-(PEG)-(PPG) block copolymer are preferable, and PEG is more preferable. The preferable PEG structure is represented by the following formula:

—(CH₂CH₂O)ₙ—,

wherein n represents an integer of 2 to 500, preferably 2 to 100, more preferably 2 to 50, and even more preferably 4 to 10.

A spacer of the present invention has a polyalkylene glycol structure. The polyalkylene glycol structure preferably binds to each biotin and Cypridina luciferase through an ester bond, amide bond or thioether bond, and preferably through amid bond.

A biotin labeling reagent, for example, having the following structure can be used:
X1—Y—(CH₂)m1—(OCH2CH2)m2—NH-(biotinyl),
X2—Y—(CH₂)m1—(OCH2CH2)m2—NH-(biotinyl),
   wherein X1 represents an active ester residue capable of forming an amide (NHCO) or aminoalkyl group by reacting with amino groups such as sulfosuccinic acid imideoxycarbonyl groups, succinic acid imideoxycarbonyl groups, tetrafluorophenoxycarbonyl groups, cyanomethyloxycarbonyl groups, p-nitrophenyloxycarbonyl groups, a halogen atom such as I, Br, or Cl, or a maleimide group; Y represents a suitable linkage group such as CH₂CONH or CH₂CH₂CONH, or a single bond; m1 represents an integer of 2, 3 or 4; m2 represents an integer of 2 to 500, preferably 2 to 100, more preferably 2 to 50, and even more preferably 4 to 10.

Examples of Biotin labeling reagents capable of introducing a polyalkylene glycol group include various types of biotin labeling reagents, for example, EZ-Link NHS-PE04 Biotinylation kit or EZ-Link TFP-PEO Biotinylation kit, which are produced by Pierce, and like commercially available kits.

The biotin labeling reaction is performed by reacting the above biotin labeling reagent and Cypridina luciferase at a temperature between 1 to 37° C., and preferably at room temperature.

Cypridina luciferase has approximately 30 lysine residues per molecule. Biotin can be introduced through the reaction of the lysine residues in Cypridina luciferase and N-hydroxysuccinimide (NHS) ester. However, conditions to avoid inactivation of luciferase must be established. Specifically, lysine is a hydrophilic residue, however, a chemically hydrophobic portion is generated on the protein surface after chemical modification. The hydrophobic portion of the enzyme affects the three-dimensional structure; therefore, biotin is effectively introduced through a hydrophilic spacer. Examples of hydrophilic spacers include alkylene residues represented by the formula:

—(CH₂CH₂)ₙ—,

wherein n represents an integer of 4 or less, preferably 3 or less, particularly 2 or 3. The alkylene residues can be linked to hetero atoms such as O, and optional hetero atom containing groups such as NH, CONH, NHCO and the like (particularly a polar group).
II. Biotin Conjugation to Tagged Cypridina luciferase

Other than the above-mentioned chemical modification method of biotynylating lysine, a method of employing a tag (peptide) can be used. According to this method, a tag is added to the C terminus or N terminus of Cypridina luciferase and the lysine in the tag is specifically biotinylated using biotin ligase. Among such commercially available tags, Avi-tag™ produced by GeneCopoeia, Inc. (LERAPGGLNDIFEAQKIEWHE (SEQ ID NO: 2) or GLNDIFEAQKIEWHE (SEQ ID NO: 3)) and BioEase Tag™ produced by Invitrogen Corporation (72 amino acid residues (peptide with amino acid residues of 524 to 595) in the a subunit of partial C terminus sequence of Klebsiella pneumoniae oxalacetic acid decarboxylase) are well known; however, such tags are not limited to these.

III. Biotinylation of Cypridina Luciferase through a Sugar Chain

Sugar-chain-linked Cypridina luciferase is obtained when Cypridina luciferase, which is a secretory sugar protein, is expressed in yeasts, animal cells (originated from insects, mammals, and the like) and the like. Reacting the sugar chain with periodate such as NaIO₄, diol groups in the sugar chain is oxidized, thereby introducing aldehyde groups into the sugar chain portion. When the resultant product is reacted with a biotinylation reagent having a group selectively reactive to an aldehyde group (for example, a hydrazide group (CONHNH₂)), biotin does not react with Lysine in Cypridina luciferase, but biotin is selectively introduced into the sugar chain. It is also possible to bind biotin by causing the aldehyde group to react with the amino group of the biotinylation reagent to produce an imine, and reducing the resulting imine using NaBH₃CN.

Introduction of biotin into the sugar chain allows biotinylation, while maintaining Cypridina luciferase activity, and is therefore preferable. Examples of periodates include sodium periodate, potassium periodate, lithium periodate, and the like.

Oxidization using a periodate such as 2 to 6 mg of NaIO₄ per 0.5 to 10 mg of a sugar chain-containing Cypridina luciferase allows oxidation and cleavage of the diol group included in the sugar chain. Use of the resulting aldehyde allows selective biotinylation of Cypridina luciferase at the sugar chain. Introduction of biotin into the sugar chain can be achieved, for example, following Scheme 1:

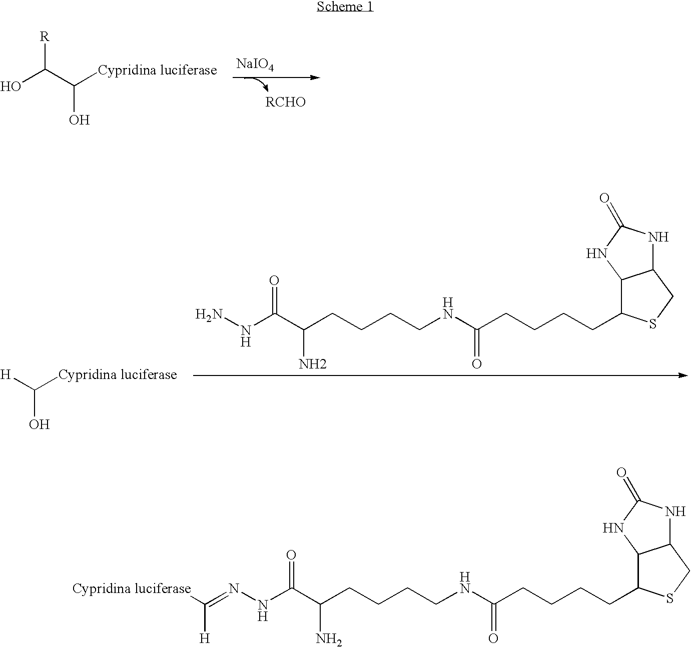

wherein R represents the functional groups of a sugar chain. Cypridina luciferase has a portion of a sugar chain.

In addition to the biotinylation reagent described above, two types of reagent, having the following formulae, are commercially available from Pierce.

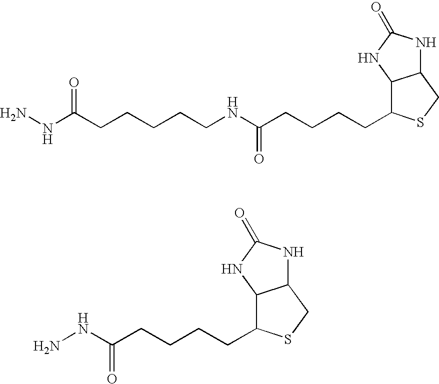

These biotinylation reagents are merely examples. It is possible to use any reagent as long as it has a group that causes biotin to react selectively with the aldehyde group.

The biotin labeled Cypridina luciferase of the present invention is bound to usually 1 to 10 biotin residues, preferably 2 to 5 residues, more preferably 2 to 3 residues per Cypridina luciferase. When excessive number of biotin residues is bound, Cypridina luciferase tends to be easily inactivated, thereby reducing the active proportion of Cypridina luciferase bound per biotin.

The biotin labeled Cypridina luciferase of the present invention forms a complex with avidin, streptavidin, NeutrAvidin® and the like. The complex is further bound to at least one of biotin-bound bioactive substance such as antibodies, antigens, DNA, proteins or peptide hormones (including a wide variety of ligands capable of recognizing cells or tissues, for example, protein hormones or peptide hormones, steroid hormone and the like) and sugar chains (for example, Sialyl Lewis X and the like). Such biotin bound substances can be used for various types of assay systems including immunoassay and the imaging of cells or tissues (especially portions that have disorders such as cancers).

Quantum dots are composed of atoms that are used in semiconductor materials. The core of commercially available quantum dots are, for example, made of sulfur layer, cadmium mixed with selenium or tellurium, or silicon as starting materials. The quantum dots may be modified by a polymer coating or the like. Such surface modified quantum dots, being further bound with polyvalent avidin substrates such as biotin and streptavidin, are well known, and are commercially available.

The quantum dot emits fluorescence light with high transparency close to red light within the range of about 500 to about 800 nm, i.e., in the near-infrared region or in the visible region of at least 500 nm, preferably at least 600 nm, more preferably at least 650 nm, particularly preferably red light of at least 700 nm. In the case of detecting the fluorescence of the quantum dot using a CCD camera, wavelengths within the range of 650 to 700 nm are preferable in consideration of the optical properties of a CCD camera. In the present invention, luminescence emitted from Cypridina luciferase causes the quantum dot to emit fluorescence, therefore, the selection of the appropriate wavelength of the quantum dot is important.

It is possible to use commercially available biotinylated quantum dots, for example, Qdot® 605 Biotin, Qdot® 655 Biotin and the like are available from Invitrogen Corporation.

A polyvalent avidin substrate can be introduced into Cypridina luciferase and bioactive substances (antibodies, peptides, organic compounds, hormones, enzyme substrates, sugar chains and nucleic acids), via appropriate linkers such as heterobifunctional-group type linkers having a succinimide group and maleimide group, or linkers having two succinimide groups. Or, when a sugar chain-containing Cypridina luciferase is used, it is treated with NaIO₄ and the like in the same manner as in Scheme 1 to introduce an aldehyde group, and a hydrazide group which reacts with the aldehyde group and a heterobifunctional group linker having an amino group (NH₂) or a thiol group (SH) that reacts with a polyvalent avidin substrate are used to link the sugar-containing Cypridina luciferase and the polyvalent avidin substrate.

In the present specification, the term “hormone” shall be understood to include substances capable of binding to the cell surface, including any substances capable of distinguishing cells through the binding, mainly including peptide/protein hormones, homing peptides and the like. Such hormones are not limited to these, including non-peptide hormones (steroid hormones, thyroid hormones and the like) as well.

The antibodies [of the present invention] are not particularly limited; however, for the imaging of cells/tissues, it is preferable to use antibodies capable of distinguishing substances presented on the cell surface.

It is possible to use commercially available quantum dots having combined therewith polyvalent avidin substrates, for example, Qdot® 605 Streptavidin, Qdot® 585 Streptavidin, Qdot® 655 Streptavidin, Qdot® 565 Streptavidin, Qdot® 525 Streptavidin, Qdot® 705 Streptavidin, Qdot® 800 Streptavidin and the like are available.

According to the present invention, the combination of Cypridina luciferase and a quantum dot enables the luminescence of visible to near-infrared light emitted from a quantum dot, thereby allowing imaging deep inside living bodies. A visible to near-infrared light-emitting complex can be prepared by making a complex of a quantum dot, which itself emits visible to near-infrared light fluorescence, and Cypridina luciferase.

For example, commercially available Qdot® 655 Streptavidin is used in Example 1. In Example 5, Streptavidin is linked to the carboxyl group on the shell of Qdot® 655 ITK™ Carboxyl Quantum Dots produced by Invitrogen Corporation. It is possible to use the following other commercially available quantum dot products:
Qdot® 605 ITK™ Carboxyl Quantum Dots, Qdot® 585 ITK™ Carboxyl Quantum Dots, Qdot® 655 ITK™ Carboxyl Quantum Dots, Qdot® 565 ITK™ Carboxyl Quantum Dots, Qdot® 525 ITK™ Carboxyl Quantum Dots, Qdot® 705 ITK™ Carboxyl Quantum Dots, Qdot® 800 ITK™ Carboxyl Quantum Dots.

It is possible to use Cypridina luciferase labeled with biotin or a polyvalent avidin substrate of the present invention, and a quantum dot labeled with biotin or a polyvalent avidin substrate for the labeling of bioactive substances. For example, such a complex is further made into a complex with a peptide capable of distinguishing antibodies or specific cells such as cancer cells and the like, and the resulting complex is applied to a living body (for example, a human with cancerous tissues), thereby allowing the imaging of specific cells/tissues (for example, cancer tissues). Such a method is effective for the perfect excision of cancer tissues.

The complex of the present invention is prepared by biotinylating any combination of two of the following substances; (1) Cypridina luciferase, (2) a quantum dot, (3) a bioactive substance, introducing a polyvalent avidin substrate into the remaining substance, and mixing the polyvalent avidin-modified substrate and the resulting two biotinylated substances together or separately. Thus, a complex of the present invention, where Cypridina luciferase, a quantum dot and a bioactive substance are linked through the binding between biotin and a polyvalent avidin substrate can be obtained. Preferable preparation methods include the following methods (i) and (ii).
(i) A polyvalent avidin substrate is introduced into a bioactive substance, which is then reacted with a biotinylated quantum dot and biotinylated Cypridina luciferase sequentially or simultaneously, leading to the preparation of a bioactive substance labeled with Cypridina luciferase and a quantum dot.
(ii) A polyvalent avidin substrate is introduced into Cypridina luciferase, which is reacted with a biotinylated quantum dot and a biotinylated bioactive substance simultaneously or sequentially. Accordingly, a complex in which a bioactive substance is labeled with Cypridina luciferase and a quantum dot is prepared complex.

### EXAMPLES

The present invention will be described in detail below with reference to Examples.

### Example 1

### Complex of Biotinylated Antibody, Biotinylated Luciferase (Biotin Enzyme) and Avidin Quantum Dot (FIG. 1)

A biotinylated mouse anti-IFN antibody solution contained in an IFNα ELISA kit available from GE Healthcare was added to an anti-mouse IgG-immobilized 96-well microplate purchased from Pierce. The microplate was gently shaken. After removing the solution, the microplate was washed with 0.15 ml of a solution of 20 mM Tris-HCl (pH 7.8), 0.9% NaCl, and 0.1% Tween 20 four times.

A 0.001 mM solution of avidinylated quantum dots (650 nm) purchased from Invitrogen Corp. was diluted with a solution of 20 mM Tris-HCl (pH 7.8), 0.9% NaCl, and 0.1% Tween 20 to a series of concentrations of 80, 40, 20, 10, 5, 2.5, 1.25 and 0.63 nM. Each of the serially diluted concentrations of the quantum dot solution was mixed with the same amount of 8.3 nM biotinylated luciferase. The mixture was allowed to stand on ice for 30 minutes. The resulting solution was added to an 8-well microplate, and the microplate was gently shaken for 15 minutes. After removing the solution, the microplate was washed with 0.15 mL of a solution of 20 mM Tris-HCl (pH 7.8), 0.9% NaCl, and 0.1% Tween 20 four times.

Then, 0.1 ml of a 1000 nM Cypridina luciferin solution was added to the 96-well microplate, and the luminescence was measured. At the concentration of 2.5 nM, the maximum emission was observed. The results show that a complex can be formed by mixing the biotinylated antibody, biotinylated enzyme, and avidin quantum dot in a molar ratio of 1:3:1.

### Example 2

### Emission Spectrum of a Complex of the Biotinylated Antibody, Biotinylated Luciferase, and Avidin Quantum Dot (650 nm)

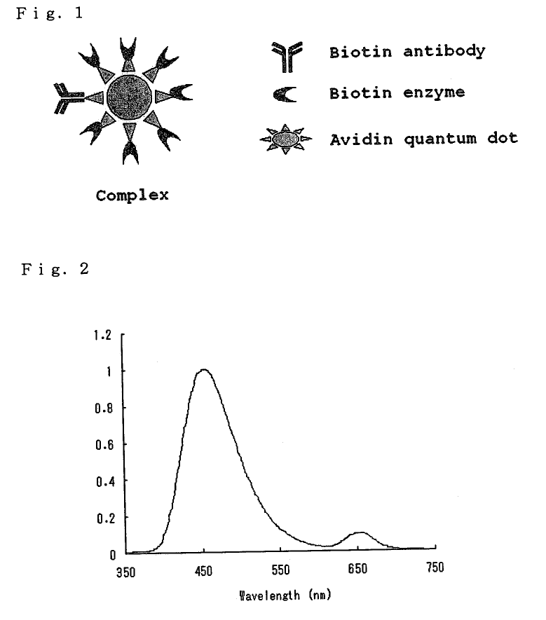

A solution containing biotinylated luciferase and avidin quantum dots (650 nm) in a molar ratio of 3:1 was prepared. After being allowed to stand for 30 minutes, the solution was diluted with a solution of 20 mM Tris-HCl (pH 7.8), 0.9% NaCl, and 0.1% Tween, and then centrifuged using a Biomax 100 k filter (product of Millipore Corporation) at 10000 g for 5 minutes three times. The residue was collected and reacted with a 1000 nM Cypridina luciferin solution, and the emission spectrum was determined. As a result, luminescence by energy transfer was observed in the longer wavelength region (FIG. 2).

### Example 3

### IFNα-ELISA Assay Using the Complex of Biotinylated Antibody, Biotinylated Luciferase, and Avidin Quantum Dot

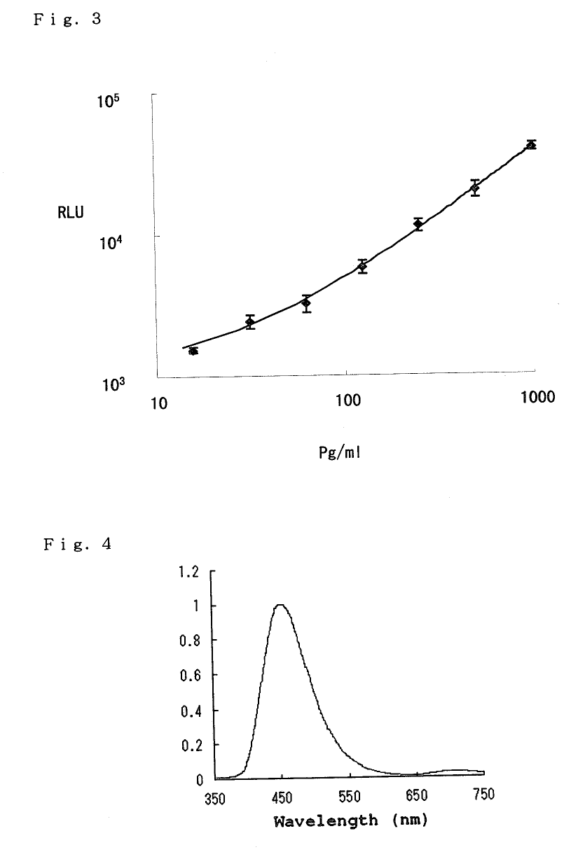

IFNα was detected using an anti-human IFNα antibody-immobilized 96-well microplate, an IFN reference standard, a biotin-labeled anti-human IFNα antibody all contained in an IFNα ELISA kit available from GE Healthcare, and using a complex of biotin-labeled Cypridina luciferase and a polyvalent avidin substance. 0.5 mL of the IFN preparation was diluted to a series of concentrations of 0, 15.6, 31.2, 62.5, 125, 250, 500, and 1000 pg/mL. 0.05 mL each of the serially diluted IFNα solutions and 0.05 mL of the biotin-labeled anti-human IFNα solution were added to three rows in the anti-human IFNα antibody-immobilized 96-well microplate. The microplate was gently shaken for 2 hours. After removing the solution, the microplate was washed with 0.15 mL of a solution of 20 mM Tris-HCl (pH 7.8), 0.9% NaCl, and 0.1% Tween 20 four times. The complex of the biotinylated antibody, biotinylated luciferase, and avidin quantum dot obtained in Example 1 was added to each well, and gently shaken for 15 minutes. After removing the solution, the microplate was washed with 0.15 mL of a solution of 20 mM Tris-HCl (pH 7.8), 0.9% NaCl, and 0.1% Tween 20 four times. Then, 0.1 mL of 1000 nM Cypridina luciferin solution was added to the 96-well microplate, and the luminescence was measured. The results show that in the diluted concentrations (15.6 to 1000 pg/mL) of IFNα, the emission signal is dose-dependent on the target molecule (FIG. 3). The results show that this complex can be used as a probe for microanalysis.

### Example 4

### Emission Spectrum of a Complex of Biotinylated Antibody, Biotinylated Luciferase and Avidin Quantum Dot (705 nm)

A solution containing biotinylated luciferase and avidin quantum dots in a molar ratio of 3:1 was prepared. After being allowed to stand for 30 minutes, the solution was diluted with a solution of 20 μM Tris-HCl (pH 7.8), 0.9% NaCl, and 0.1% Tween, and then centrifuged using a Biomax 100 k filter (product of Millipore Corporation) at 10000 g for 5 minutes three times. The residue was collected and reacted with a 1000 nM Cypridina luciferin solution. The emission spectrum was measured. As a result, luminescence by energy transfer was also observed in the near-infrared region (FIG. 4).

### Example 5

### Emission Spectrum of a Complex of Biotinylated Antibody, Biotinylated Luciferase and Avidin Quantum Dot (650 nm) Obtained Using Natural Luciferin or Luciferin Analog

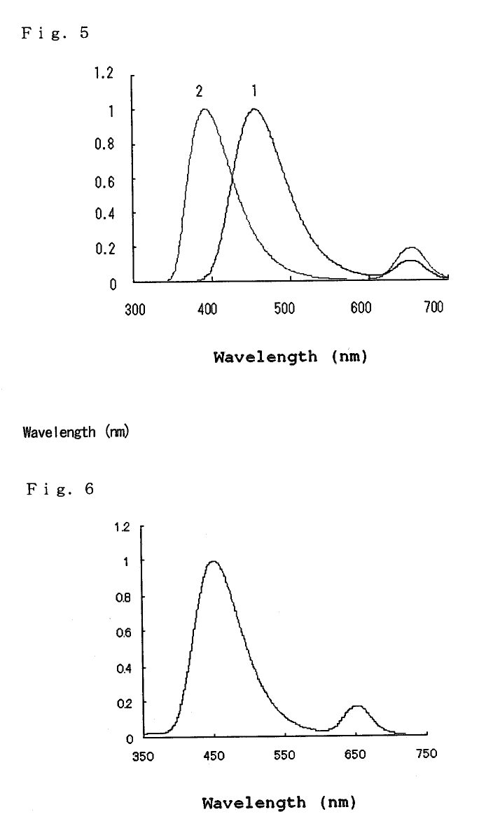

A solution containing biotinylated luciferase and avidin quantum dots (650 nm) in a molar ratio of 3:1 was prepared. After being allowed to stand for 30 minutes, the solution was diluted with a solution of 20 mM Tris-HCl (pH 7.8), 0.9% NaCl, and 0.1% Tween, and then centrifuged using a Biomax 100 k filter (product of Millipore Corporation) at 10000 g for 5 minutes three times. The residue was collected and reacted with a 1000 nM Cypridina luciferin solution or a 1000 nM Cypridina luciferin analog solution (compound 1 shown below) with an emission maximum in the lower wavelength region (λmax=390 nm). Each emission spectrum was measured. The results show that the BRET ratio of the analog is higher (FIG. 5).

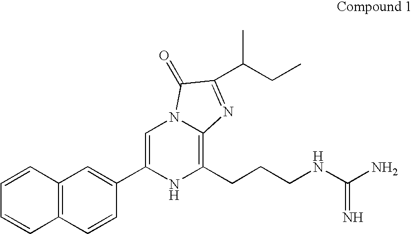

A process for preparing compound 1 is described below.

Pd(PPh₃)₄ (40 mg) was added to a solution of saturated Na₂CO₃ (24 mL) and 50 mL of dioxane containing a known compound 2 (0.2 g, 0.6 mmol) as shown below and 2-Naphthyl-boronic acid (103 mg, 0.6 mmol). The mixture was heated at 80° C. for 1 hour. After cooling, the reaction mixture was diluted with ethyl acetate, then washed with a saline solution, and dried over sodium sulfate. After filtration, the filtrate was concentrated to give a crude extract. The crude extract was purified by chromatography to give the intended product. The obtained compound was dissolved in 1 mL of a TFA (trifluoroacetic acid) solution, and stirring was continued for 1 hour, followed by concentration. The trifluoroacetate compound was dissolved in 3.5 mL of DMF, and pyrazole-1-carboxamidine (280 mg) and DIEA (301 mg) were added and stirred at room temperature overnight. Ether was added to give a precipitate. The precipitate was dissolved in methanol, and recrystallized to give a compound (3) (135 mg, 70%).

¹H NMR (500 MHz, CD₃OD) 2.21 (2H, quintet, J=7 Hz), 2.90 (2H, T, J=7 Hz), 3.37 (2H, T, J=7 Hz) 7.46 (2H, d, J=7 Hz), 7.50 (1H, d, J=7 Hz), 7.86 (2H, d, J=7 Hz), 8.01 (1H, d, J=7 Hz), 8.30 (1H, s), 8.37 (1H, s).

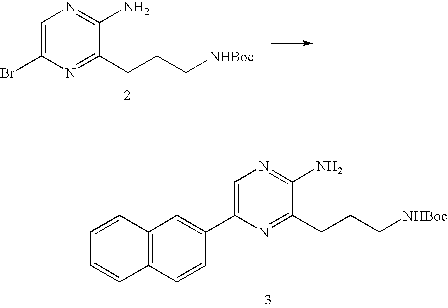

The compound (3) (10 mg), water (0.1 ml), ketoacetal, ethanol (0.1 ml), and 49% HBr (0.01 ml) were placed into a round-bottomed flask and heated at 100° C. for 1 hour. The reaction mixture was cooled to room temperature, and then concentrated under reduced pressure. The residue was dissolved in ethanol, and purified using a LH-20 column to give compound 1 (3.5 mg, 27%).

¹H NMR (500 MHz, CD₃OD) 0.96 (3H, t, J=7 Hz), 1.47 (3H, D, J=7 Hz), 1.86 (2H, M), 2.35 3.19 (1H, M) (2H, Quintet, J=7 Hz) 3.43 3.48 (2H, T, J=7 Hz) (2H, T, J=7 Hz) 7.55 (2H, d, J=7 Hz), 7.90 (1H, d, J=7 Hz), 8.01 (2H, d, J=7 Hz), 8.14 (1H, d, J=7 Hz), 8.60 (1H, s), 8.78 (1H, s).

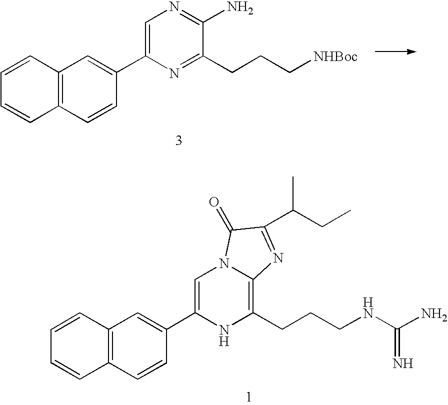

### Example 6

### Biotinylation of Sugar Chain of Cypridina Luciferase

0.1 mg of purified luciferase (relative total emission activity: 3.6×10⁸ counts) was dissolved in 0.05 mL of 0.1M acetate buffer (pH 5.2). The solution was mixed with the same amount of a solution of 20 mM NaIO₄ in 0.1M acetate buffer, and slowly stirred at 4° C. for 0.5 hours. The reaction mixture was placed in a PD-10 column (product of GE Healthcare), and eluted with a solution of 100 mM sodium phosphate and 150 mM NaCl. Only the active fractions (about 2 mL) were collected. 2 mL of the active fractions were concentrated to about 0.02 mL using a Biomax 10 k filter (product of Millipore Corporation), and mixed and reacted with 0.02 mL of a solution of 10 mM biotin hydrazide (Pierce) in 0.1 M acetate buffer (pH 5.2) at room temperature for 2 hours. The reaction mixture was placed in a PD-10 column (product of GE Health), and eluted with a solution of 100 mM sodium phosphate and 150 mM NaCl. Only the active fractions were collected (about 2 mL). 0.01 mL of the eluant diluted 1000 times and 0.05 mL of 0.001 mM luciferin were mixed to determine the luciferase activity, and a relative total emission activity of 6.6×10⁷ counts was obtained.

Solutions containing luciferase with a biotin-labeled sugar chain and avidin quantum dots (650 nm or 705 nm) in a molar ratio of 3:1 were prepared. After being allowed to stand for 30 minutes, the solutions were diluted with a solution of 20 mM Tris-HCl (pH 7.8), 0.9% NaCl, and 0.1% Tween 20, and then centrifuged using a Biomax 100 k filter (product of Millipore Corporation) at 10000 g for 5 minutes three times. The residue was collected and reacted with a 1000 nM Cypridina luciferin solution. The emission spectra were determined (FIG. 6).

1. A bioactive substance labeled with Cypridina luciferase and a quantum dot, the bioactive substance being at least one species selected from the group consisting of antibodies, peptides, organic compounds, hormones, enzyme substrates, sugar chains and nucleic acids.

2. A labeled bioactive substance according to claim 1, wherein the bioactive substance, the Cypridina luciferase and the quantum dot form a complex through binding of an avidin substrate and biotin.

3. A biological imaging method, comprising applying a bioactive substance labeled with Cypridina luciferase and a quantum dot to a living body, the bioactive substance being at least one species selected from the group consisting of antibodies, peptides, organic compounds, hormones, enzyme substrates, sugar chains and nucleic acids.

4. A method of producing a bioactive substance complex labeled with Cypridina luciferase and a quantum dot, comprising the steps of preparing a complex of a polyvalent avidin substrate and a bioactive substance, and adding a biotinylated quantum dot and biotinylated Cypridina luciferase.

5. A method of producing a bioactive substance complex labeled with Cypridina luciferase and a quantum dot, comprising the steps of preparing a complex of a polyvalent avidin substrate and Cypridina luciferase, and adding a biotinylated quantum dot and a biotinylated bioactive substance.

6. A cell labeling complex, comprising a Cypridina luciferase-bioactive substance fusion protein and a quantum dot, the bioactive substance being at least one species selected from the groups of an antibody, a peptide, an organic compound, a hormone, an enzyme substrate, a sugar chain and a nucleic acid.


SEQUENCE LISTING (3 sequences)

SEQ ID NO: 1 - protein, 636 aa, Cypridina
MRFPSIFTAVLFAASSALAALVNTTTEDETAQIPAEAVIGYSDLEGDFDVAVLPFSNSTNNGLLFINTTIASIAAKEEGVSLEKREAEAQDCPYEPDPPNTVPTSCEAKEGECIDSSCGTCTRDILSDGLCENKPGKTCCRMCQYVIECRVEAAGWFRTFYGKRFQFQEPGTYVLGQGTKGGDWKVSITLENLDGTKGAVLTKTRLEVAGDIIDIAQATENPITVNGGADPIIANPYTIGEVTIAVVEMPGFNITVIEFFKLIVIDILGGRSVRIAPDTANKGMISGLCGDLKMMEDTDFTSDPEQLAIQPKINQEFDGCPLYGNPDDVAYCKGLLEPYKDSCRNPINFYYYTISCAFARCMGGDERASHVLLDYRETCAAPETRGTCVLSGHTFYDTFDKARYQFQGPCKEILMAADCFWNTWDVKVSHRNVDSYTEVEKVRIRKQSTVVELIVDGKQILVGGEAVSVPYSSQNTSIYWQDGDILTTAILPEALVVKFNFKQLLVVHIRDPFDGKTCGICGNYNQDFSDDSFDAEGACDLTPNPPGCTEEQKPEAERLCNSLFAGQSDLDQKCNVCHKPDRVERCMYEYCLRGQQGFCDHAWEFKKECYIKHGDTLEVPDECKGSGSGSHHHHHH

SEQ ID NO: 2 - protein, 21 aa, Artificial Seuquence
  Tag
LERAPGGLNDIFEAQKIEWHE

SEQ ID NO: 3 - protein, 15 aa, Artificial Sequence
  Tag
GLNDIFEAQKIEWHE